(19) United States
(12) Patent Application Publication
(10) Pub. No.: US 2006/0047202 A1
(43) Pub. Date: Mar. 2, 2006

(54) Method and system of breathing therapy for reducing sympathetic predominance with consequent positive modification of hypertension

(76) Inventor: Stephen Bennett Elliott, Allen, TX (US)

Correspondence Address: Stephen Bennett Elliott, 702 Buffalo Springs Drive, Allen, TX, 75013, US

(21) Appl. No.: 10/932,636

(22) Filed: Sep. 2, 2004

(51) Int. Cl. A61B 5/02; A61B 5/08

(52) U.S. Cl. 600/485; 128/905; 600/500; 600/529

(57) ABSTRACT

The invention specifies a method and system for leading a person suffering from “sympathetic predominance”, a specific symptom of which is “hypertension”, to breathe according to a certain pattern for the express purpose of positively altering the condition of sympathetic predominance (over activation), having the effect of bringing the autonomic nervous system into the state of balance, with consequent reductions in “tenseness”, blood pressure, muscular tightness, and emotional strain, as well as the alleviation of the myriad of subtle neuro-physiological consequences resulting from sympathetic predominance potentially including headaches, anxiety, sleep disorders, allergies, and other maladies that have yet to be attributed to this condition, thus leading to a general improvement in health, well being, and homeostasis. It accomplishes this by systematically reducing the breathing frequency with consequent increases in breathing depth, the ultimate goal being the realization and ongoing maintenance of 1 complete breathing cycle in 11.76 seconds or 5 complete breathing cycles in 58.8 seconds, the result being improved health, well being, and homeostasis.

### RELATED PATENT FILINGS

Method and System for Consciously Synchronizing the Breathing Cycle with the Natural Heart Rate Cycle (10/699,025), System and Method for Synchronizing the Heart Rate Variability Cycle With The Breathing Cycle (Feb. 19, 2004), Method of Presenting Audible and Visual Cues for Synchronizing the Breathing Cycle With An External Timing Reference for Purposes of Synchronizing The Heart Rate Variability Cycle With The Breathing Cycle (Mar. 15, 2004), Method and System Providing A Fundamental Musical Interval for Heart Rate Variability Synchronization (Mar. 23, 2004), Method and System of Respiratory Therapy Employing Heart Rate Variability Coherence (10/814,035).

### FIELD OF THE INVENTION

The present invention relates to the field of human health and in particular to what is a potentially a new field of therapy with the specific purpose of preventing or reducing sympathetic predominance, “sympathetic predominance” referring to over-activation of the sympathetic branch of the autonomic nervous system and the relative under activity of the parasympathetic branch, and positively modifying its resultant conditions, one of which is proposed to be “hypertension”.

The reason that it is a potentially new field of therapy is that, while it involves “breathing” it is not “respiratory therapy” in the traditional sense, for it concerns itself with the matter of blood gases only indirectly. Neither is it a present concern of “physical therapy”. The present invention, defines a specific form of therapy wherein breathing is employed in order to realize fundamental changes in neuro-physiological functioning, specifically, positive modification of autonomic nervous system function, or more specifically, the correction of sympathetic nervous system predominance, one of its resultant conditions being “hypertension”.

Consequently, for purposes of this patent, said therapy will be referred to as “breathing therapy”.

### BACKGROUND OF THE INVENTION

Hypertension or “high blood pressure” is presently defined as “a medical condition in which constricted arterial blood vessels increase the resistance to blood flow, causing the blood to exert excessive pressure against vessel walls”.¹ It is also recognized that “two factors determine blood pressure: the amount of blood the heart pumps and the diameter of the arteries receiving blood from the heart. When the arteries narrow, they increase the resistance to blood flow. The heart works harder to pump more blood to make sure the same amount of blood circulates to all the body tissues. The more blood the heart pumps and the smaller the arteries, the higher the blood pressure. As a measure of overall heart function doctors use cardiac output, the amount of blood pumped by each ventricle in one minute. Cardiac output is equal to the heart rate multiplied by the stroke volume, the amount of blood pumped by a ventricle with each beat. Stroke volume, in turn, depends on several factors: the rate at which blood returns to the heart through the veins, how vigorously the heart contracts, and the pressure of blood in the arteries, which affects how hard the heart must work to propel blood into them. An increase in either heart rate or stroke volume—or both—will increase cardiac output.”¹ In summary, the higher the cardiac output, the higher the blood pressure.
(¹Microsoft Encarta, Microsoft Corporation)

Relative to central nervous system functioning, hypertension is the state wherein the sympathetic (activating) function has persistent predominance over the parasympathetic (deactivating) function. It is sympathetic action that elicits accelerated heartbeat rate and contractile vigor. In theory, sympathetic action also governs blood vessel constriction; these factors combined, resulting in the state of hypertension.

Hypertension represents a huge health care challenge where large percentages of the adult, and now adolescent population, are identified as being hypertensive. Greater than 25% of the American population is estimated to be affected by hypertension. Hypertension is known to be strongly related to cardiopulmonary integrity, stroke, and internal organ health. Today, the treatment of hypertension is approached through the application of pharmaceuticals, diet, fitness, and lifestyle modification. “If these (lifestyle modification) methods do not correct hypertension, a physician may prescribe medications known as antihypertensives. Diuretics are antihypertensives that promote excess salt and water excretion, reducing the amount of fluid in the bloodstream and relieving pressure on blood vessel walls. Beta blockers reduce heart rate and the amount of blood the heart pumps. ACE inhibitors prevent the narrowing of blood vessel walls to control blood pressure. Calcium channel blockers slow heart rate and relax blood vessels.”¹ While these drugs are effective for some, they are non-effective for others, also often presenting negative side effects, sometimes severe. For many people, their hypertension continues, ultimately reducing their well being, increasing their risk of serious disease, and reducing their longevity.
(¹Microsoft Encarta, Microsoft Corporation)

The cost of hypertension including human costs, healthcare system costs, and pharmaceuticals runs into the $B per annum in the United States alone. It is generally assumed that hypertension is a necessary condition of modern life.

Research on which this patent is based, strongly indicates that a root cause (if not the root cause) of hypertension is in fact “inadequate breathing”. Inadequate breathing results in sympathetic nervous system predominance with a like withdrawal of parasympathetic action. FIG. 1 depicts the heart rate variability patterns and average heartbeat rates of a resting test subject breathing at 4 different breathing rates: 5 breaths per minute A, 7.5 breaths per minute B, 15 breaths per minute C, and 30 breaths per minute D. As can be seen:
1) Heart rate variability (amplitude) shrinks as breathing frequency increases.
2) The average heartbeat rate shifts upward as breathing frequency increases. These measurements are taken while the subject is at rest. This behavior is consistent with the behavior of the cardiopulmonary system during exercise, i.e., during exercise, the cardiopulmonary system accelerates to address the demand for increased oxygen, yet in the state of rest there is no increasing oxygen demand, except for a slight increase as a consequence of increased diaphragmatic activity. Why and how the average heartbeat rate increases with increased breathing frequency while in the resting state is not fully understood.
3) Contrasting 5 breaths per minute with 30 breaths per minute, 30 breaths per minute results in the heart working much faster on a continuous basis than 5 breaths per minute. To be clear, at 30 breaths per minute, the heartbeat rate varies between ˜91 and ˜93 BPM, never slowing down below ˜91 BPM. At 5 breaths per minute the heartbeat rate varies between ˜60 and 94 BPM, 50% of the time it is below 77 BPM and 88% of the time it is below 91 BPM. Consequently, if we compare these two “linearly”, relative to 30 breaths per minute, 5 breaths per minute allows the heart rest for 88% of the time, i.e. for 88% of the time the heartbeat rate is less than ˜91 BPM.

Per the prior discussion, heartbeat rate is one factor that directly affects blood pressure, such that, as the heartbeat rate increases, blood pressure increases. Consequently, it clearly follows that faster shallower breathing, even while at rest, increases heartbeat rate and blood pressure and slower deeper breathing reduces heartbeat rate and blood pressure.

Most people breathe at a rate of 10-15 breaths per minute.² While 30 breaths per minute was used in the prior example for contrast, the same basic relationship holds true for the range 10-15 breaths per minute. If we compare 5 breaths per minute with 15 breaths per minute, respective average heartbeat rates are 77 vs. 86, with heart rate variabilities ranging from 60-94 vs. 84-88 BPM. Comparing these two “linearly”, relative to 15 breaths per minute, 5 breaths per minute allows the heart rest for 70% of the time, i.e. for 70% of the time the heartbeat rate is less than ˜84 BPM.

The cardiopulmonary system of a human adult in a resting or semi-active state aspires to a specific resting frequency of 0.085 cycles per second or 5 cycles in ˜1 minute. At this rate, the cardio pulmonary system is optimally effective and efficient heart rate variability being of maximal amplitude, periodicity, and coherence, i.e. free of distortion. The heartbeat rate at this breathing rhythm, in this case 77 beats per minute, defines the autonomic baseline above which the sympathetic function is predominant and below which the parasympathetic function is predominant Referring once again to FIG. 1, this breathing frequency is characterized by the line titled “fundamental quiescent rhythm”. Again, for this test subject, breathing at this rate, yields an average heartbeat rate of 77 beats per minute, 77 BPM being the baseline between sympathetic and parasympathetic emphasis. In other words, relative to this test subject, an instantaneous heartbeat rate above 77 BPM represents sympathetic (activating) emphasis and an instantaneous heartbeat rate below 77 BPM represents parasympathetic (deactivating) emphasis. Consequently, as the average heartbeat rate shifts upward (above 77 BPM) as a consequence of breathing at a pace exceeding ˜5 breaths per minute, the autonomic nervous system shifts from the state of balance, sympathetic and parasympathetic equality, toward sympathetic predominance. The further it shifts in the positive direction, the stronger the sympathetic dominance. For this subject, the relationship between the average heartbeat and breathing rate is quite linear above 7.5 breaths per minute, varying at 3 beats per 7.5 breaths as detailed in FIG. 2.

FIG. 2 makes clear the fact that while average heartbeat rate varies only slightly across a relatively wide range of breathing frequencies, heart rate variability varies widely.

The inventor asserts that breathing at a rate above 5 breaths in 58.8 seconds, while at rest, if persistent, results in the pathological condition of “sympathetic predominance” or sympathetic over activation and parasympathetic under activation. Consequently, that the typical breathing rate of 10-15 breaths per minute produces the condition of sympathetic over activation in much of the population predisposing said population to a myriad of maladies, one of which is the class of symptoms commonly referred to as “hypertension”.

In summary, it is the premise of this patent, that:
1) The average heartbeat rate at the fundamental quiescent rhythm of 1 complete breathing cycle in 11.76 seconds or 5 complete breathing cycles in 58.8 seconds defines the baseline between sympathetic and parasympathetic emphasis on an individual basis. This is generally true for the adult population.
2) A second premise is that breathing at a rate faster than the fundamental quiescent rhythm of 1 complete cycle in 11.76 seconds directly results in the state of autonomic imbalance, specifically sympathetic predominance or over activation, and a corresponding parasympathetic withdrawal or under activation. This is also generally true for the adult population.
3) A third premise is that upwardly shifting average heartbeat rate and shrinking heart rate variability coincident with increasing breathing frequency, is an accurate indicator of sympathetic over emphasis, which, if persistent, results in a pathological neuro-physiological status, specifically including “hypertension”.
4) A fourth premise is that autonomic balance can be regained by breathing at slower rates, the ideal rate being the fundamental quiescent rhythm of 1 complete cycle in 11.76 seconds or 5 complete cycles in 58.8 seconds. Breathing at rates below ˜5 breaths per minute has proven to be non-productive, resulting in distortion of the heart rate variability pattern.
5) A fifth and final premise is that sympathetic predominance can averted and its affects avoided by adopting a “normal” breathing frequency of 1 complete cycle in 11.76 seconds or 5 cycles in 58.8 seconds.

### SUMMARY OF THE INVENTION

The invention specifies a system and method for leading a person suffering from “sympathetic predominance”, a specific symptom of which is “hypertension”, to breathe according to a certain pattern for the express purpose of positively altering the condition of sympathetic predominance (over activation), having the effect of bringing the autonomic nervous system into the state of balance, with consequent reductions in “tenseness”, blood pressure, muscular tightness, and emotional strain, as well as the alleviation of the myriad of subtle neuro-physiological consequences resulting from sympathetic predominance potentially including headaches, anxiety, sleep disorders, allergies, and other maladies that have yet to be attributed to this condition, thus leading to a general improvement in health, well being, and homeostasis.

An instructive method is specified for both therapy practitioners and care recipients in the application of the preferred embodiments of the present invention to the general condition of sympathetic predominance as is elicited by inadequate breathing, and the specific symptomology commonly referred to as “hypertension”.

This patent represents new art relative to the application of “breathing therapy” to the resolution of the general condition of autonomic nervous system imbalance, specifically the condition of sympathetic predominance or over activation and parasympathetic under activation. A general definition is provided relative to the objective “ideal” state of autonomic balance and how this state is achieved and maintained. Specific focus is provided as to how to correct the state of predominance, once identified. Application of the present invention to the symptoms commonly referred to as “hypertension” is described. As the correction of sympathetic predominance via breathing therapy is a nascent field of investigation, it is anticipated that it will find broad application in the alleviation of numerous maladies that are rooted in sympathetic over activation. Those skilled in the art will recognize that those applications are considered within the scope of the concepts disclosed herein and the claims that follow. Application of the present invention may be employed alone or in combination with medication as is deemed appropriate by the attending health care professional.

### BRIEF DESCRIPTION OF THE DRAWING FIGURES

The accompanying drawing figures incorporated in and forming a part of this specification illustrate several aspects of the invention and together with the description serve to explain the principles of the invention.

FIG. 1 presents a graphical model of 4 breathing rates, presenting resultant average heartbeat rates and heart rate variability patterns.

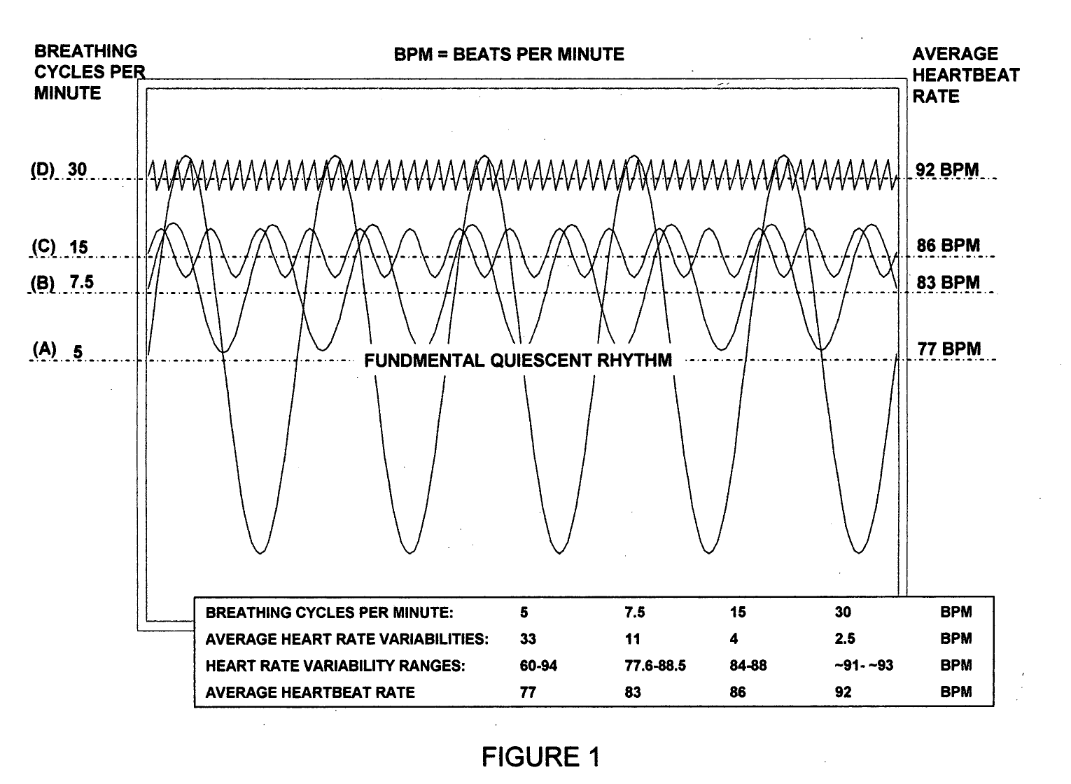

FIG. 2 presents a graph depicting the 4 breathing rates of FIG. 1 presented along a linear scale. Heart rate variability ranges at each of the 4 breathing rates are also depicted.

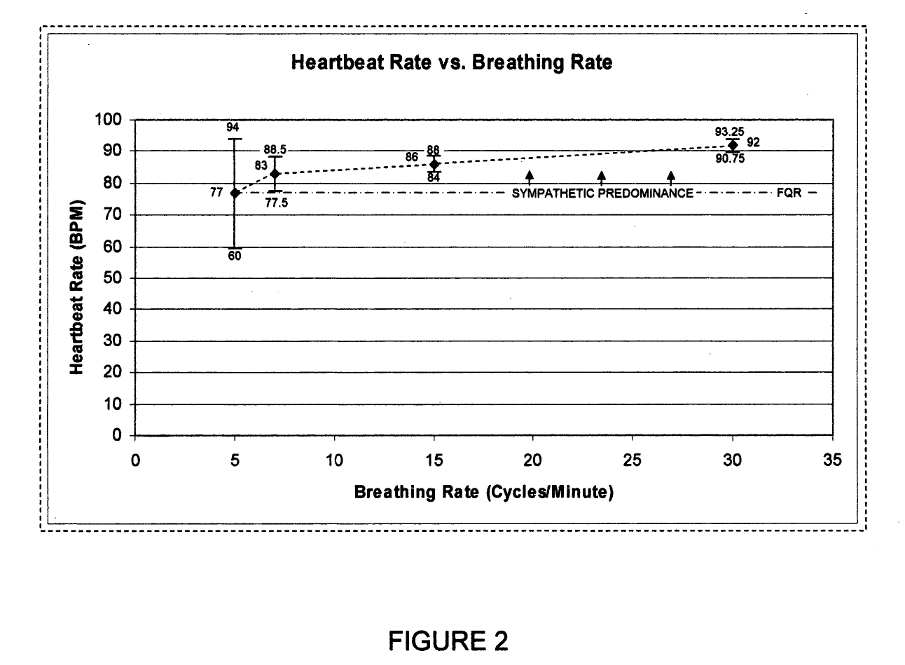

FIG. 3 presents a block diagram of one preferred embodiment of the present invention for relatively stationary applications.

FIG. 4 presents a table detailing breathing cycle programmability steps and associated breathing intervals. Track numbers for compact disk or digital video disk application are also specified.

FIG. 5 presents a second preferred embodiment wherein an “integrated training and monitoring system” is provided.

FIG. 6 describes programmability aspects of the integrated training and monitoring system of FIG. 5.

FIG. 7 provides a logical description of the basic control systems of preferred embodiments as described in FIGS. 3 and 5.

### DETAILED DESCRIPTION OF THE PREFERRED EMBODIMENTS

The present invention provides a method and system by which “breathing therapy” may be optimally applied to a conscious recipient or recipients by facilitating the slowing of the recipients resting breathing rate to the ultimate rhythm of 1 cycle in 11.76 seconds, inhalation persisting for 5.88 seconds and exhalation persisting for 5.88 seconds. Additionally, several sub-methods and sub-systems are defined providing alternative means of presenting the recipient with breathing cues and for monitoring the breathing rate of the recipient in both stationary and mobile (normal walk of life) settings.

The care recipient is presented an audio, visual, or audio-visual representation of the objective breathing cycle with a gradually increasing interval (decreasing frequency) to which the recipient consciously synchronizes their breathing cycle. In this way, a person suffering from chronic sympathetic predominance might start out with a pathological breathing frequency of 20 cycles per second, 20 cycles per second being used for example only, and over some time of training, gradually lower their “normal” breathing frequency to 15, to 10, and eventually to 5 cycles in approximately 1 minute. Instruments for monitoring the breathing cycle are applied for “feedback” purposes in the early stages of training and for ongoing monitoring relative to acute scenarios. Relative to the treatment of hypertension, the subject's blood pressure is gauged regularly as they progress from a higher breathing frequency to a relatively lower frequency over some duration of training.

A stepwise approach is specified because it is typically impractical for a person suffering from chronic sympathetic predominance to radically alter their breathing pattern all at once. A primary reason for this is that in order to breathe slower, one must also breathe deeper requiring conscious coordination and control. Breathing deeper requires the employment of the diaphragm and intercostal muscles. As is true with learning any new physical skill, it takes time to learn to coordinate the movement as well as tonify and build the respective muscle groups that are involved. This is especially true of the diaphragm because it is a relatively large muscle of which most people tend to have little awareness.

Once the subject reaches either their the target breathing frequency of ˜5 cycles in 1 minute, or in the case of application to hypertension, their target blood pressure, they may shift to a maintenance regimen wherein the invention is employed for ongoing reinforcement of the desired breathing frequency.

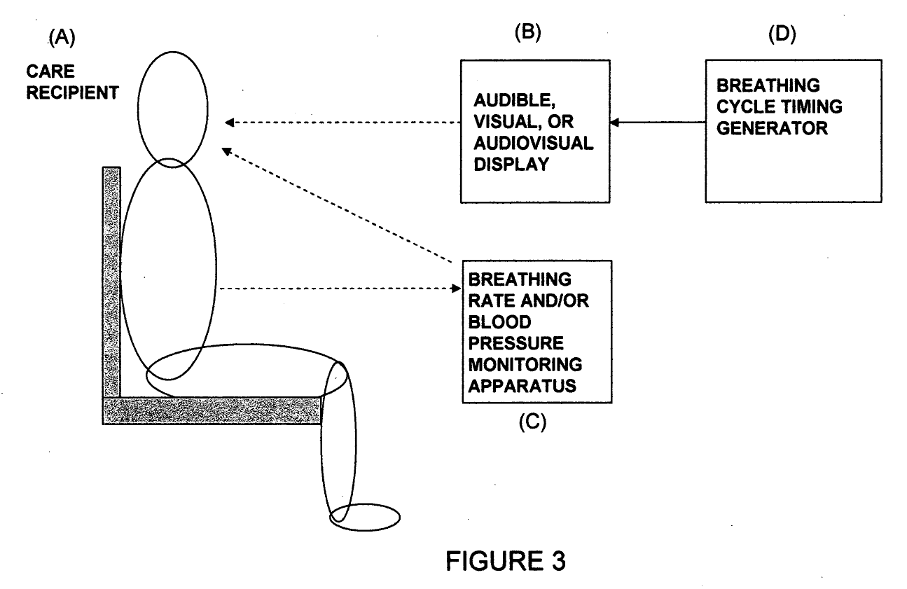

FIG. 3, specifies the preferred embodiment of the present invention in the stationary setting as might take place in a home, office, or health care setting.

While a specific instructive method is specified later, a brief discussion of the method is required here for context. Care recipient A, is positioned such that they are able to see or hear audible, visual, or audiovisual display device B. Optionally, care recipient A or a health care practitioner, attaches breathing rate and/or blood pressure monitoring apparatus C to care recipient A. Care recipient A, is able to perceive the status of their breathing rate and blood pressure as monitored by apparatus C. Upon assessing the present breathing status of care recipient A, care recipient A or alternatively, a health care practitioner, turns on breathing cycle timing generator D and selects the optimal breathing interval at which care recipient A is to practice breathing. This interval is generated by breathing cycle timing generator D and is displayed on display device B, according to the preferred mode of operation and or the ability of the given display device to support multiple forms of media. In its simplest form display device may be a speaker or set of headphones, in it's most complex form a personal computer.

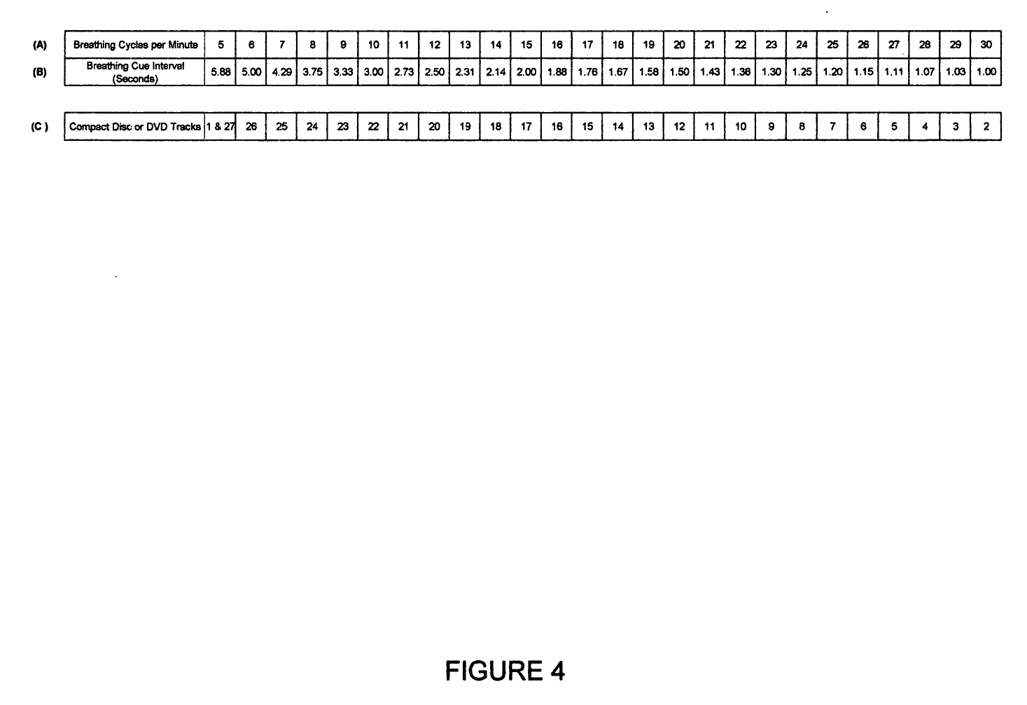

FIG. 4 provides a table defining the breathing intervals supported by breathing cycle timing generator of FIG. 3-D, ranging from ˜5 breaths per minute to 30 breaths per minute in 1 breath per minute intervals. This is depicted by row A of the table. If it is determined that the care recipient present interval is 20 breaths per minute, a setting of “18” might be selected for practice. Once care recipient A is able to breathe comfortably at “18” breaths per minute, a lower setting, for example “15” breaths per minute might be selected. The rate at which a given care recipient is able to progress toward the optimal breathing rate of 5 cycles in ˜1 minute has to do with their level of comfort, health, fitness, and extent of practice. FIG. 3, row B specifies audible, visual, or audiovisual intervals. Using 10 breaths per minute as an example, the interval for 10 breaths per minute is equal to “3”. Consequently, every 3 seconds, an audible, visual, or audiovisual indication is provided to the care recipient. This signal indicates when to inhale or when to exhale, inhalation being followed by exhalation, and visa versa.

Returning to the discussion of FIG. 3, breathing cycle timing generator D may also vary in functionality and complexity. In its simplest form it is an audio recording of varying interval played on a compact disc (CD) or MP3 player, in its visual form, a video tape or digital video disc (DVD) played on a VHS or a DVD player, and in it's most complex form a software program that is digitally generating the respective intervals on a personal computer (PC), laptop, palmtop, cellular telephone, or like device wherein a microprocessor exists to digitally synthesize audio signals containing the target intervals. In the form of a CD or DVD, multiple tracks are provided, one track supporting each breathing frequency of interest. These tracks may be repeated or played sequentially depending on the length of the track and the length of practice required. Display B and breathing cycle timing generator D may be discrete or integrated into a single element, an example of which is a personal computer. Of course the potential exists to create a purpose built microprocessor or integrated circuit based device for programmatically generating the required audible, visual, or audiovisual outputs.

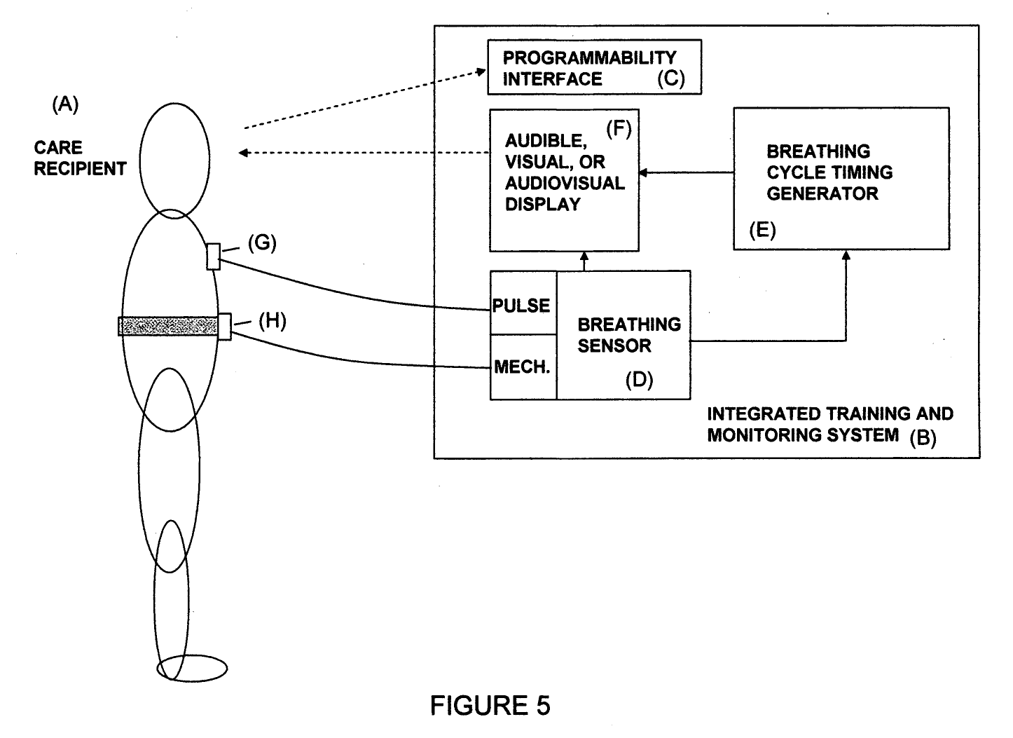

Referring now to FIG. 5, a second preferred embodiment of the present invention is the “integrated training and monitoring system” that can be carried and applied in normal walks of life. This embodiment allows a person to both practice their breathing skill as well as monitor themselves for the purpose of identifying those times when their breathing rate increases above the desired range such that corrective action may be taken in the moment. This is useful to those that desire to reinforce a new breathing behavior as well as for those whose present health status requires that they take immediate action to maintain a relatively low breathing rate, for example a person recovering from a stroke. A complete discussion of the operation of this embodiment follows.

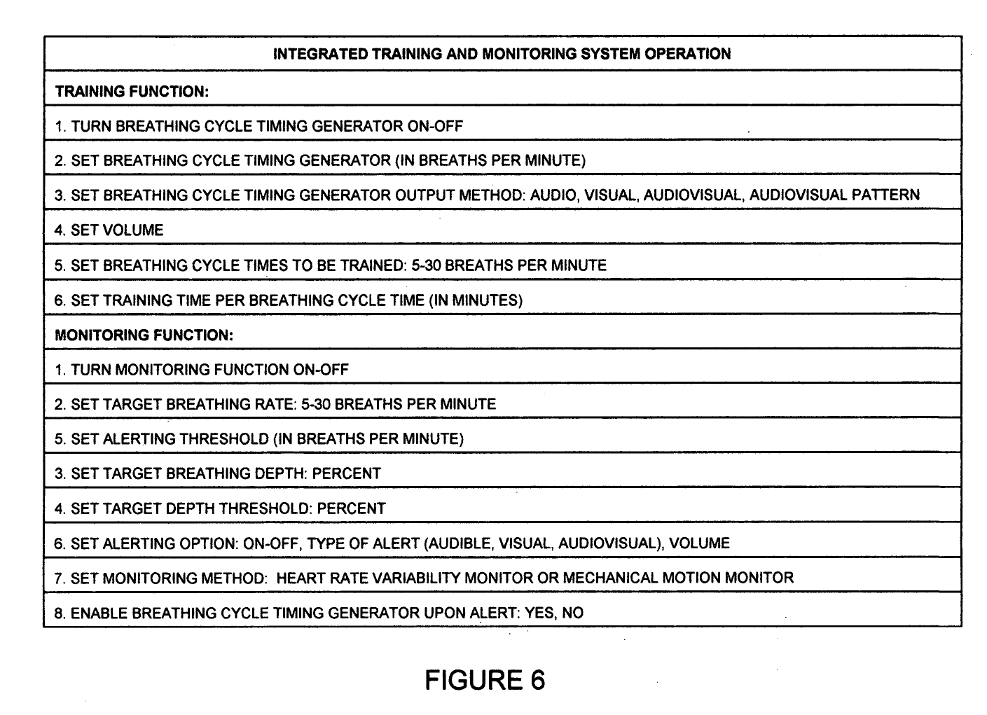

Care recipient A, is fitted with the integrated training and monitoring system of FIG. 5. This system may take numerous forms depending on packaging format and extent of integration. This may take the form of an instrument placed in the pocket, hung on the belt, worn on the wrist, or other. The recipient is fitted with a monitoring apparatus of either a pulse G, or mechanical motion H type. The mechanical motion monitor may fitted around torso with at belt assembly such that it detects the expansion and contraction of the torso with breathing. The pulse monitor may be attached to the earlobe, a finger, the wrist, etc. The unit is turned on. When so enabled, breathing sensor D begins monitoring the breathing frequency and depth on a continuous basis, frequency being a function of period and depth being a function of amplitude. If at any time, the breathing frequency or depth exceeds limits, an alert is provided. Depending on the options selected, as detailed in FIG. 6, upon the alert, the training function of the unit principally consisting of breathing cycle timing generator E and audible, visual, or audiovisual display F, is initiated resulting in the presentation of an audible, visual, or audiovisual signal to which the care recipient is to synchronize their breathing. This signal is of a lower frequency that the present rate of breathing and intended to guide the recipient back to a viable breathing frequency and lower state of sympathetic activity. This signal continues until the breathing frequency falls below the specified threshold. This has the effect of modifying the tendency toward sympathetic predominance in the moment, the result being the maintenance of a relatively lower heartbeat rate and resultant blood pressure.

Throughout the day, in the absence of an alert, the care recipient may turn on the training function of the device, principally involving breathing cycle timing generator H and audio, visual, or audiovisual display F, and practice breathing at the target rate, this having been preestablished per the instructive method detailed later.

The integrated training and monitoring system B, consists principally of programmability interface C, breathing sensor D, breathing cycle timing generator E, and display F. Breathing sensor D, supports two sensing options, pulse monitor G, via which the heart rate variability signal can be derived for purposes of determining breathing rate and depth, and mechanical sensor H, which senses the contraction and expansion of the torso commensurate with frequency and depth of breathing. Programmability aspects of programmability interface C are detailed in FIG. 6.

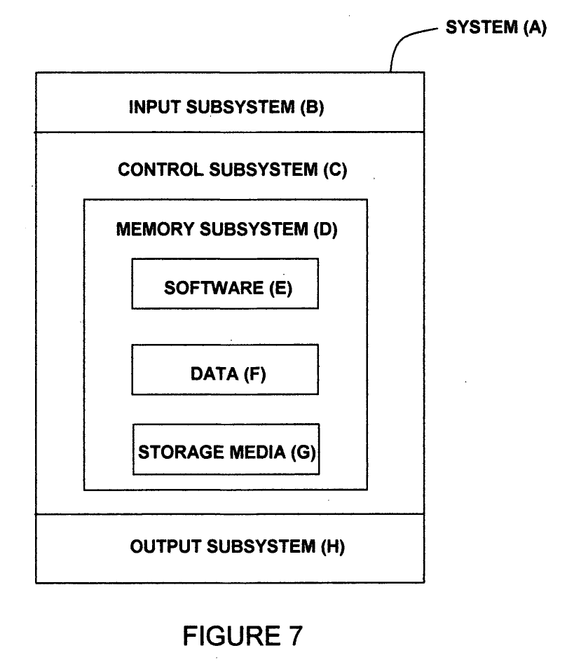

FIG. 7 provides a logical description of the basic control systems of preferred embodiments as described in FIGS. 3 and 5. Control subsystem C may be implemented in hardware, software, or hardware and software and may employ a microprocessor, microcontroller, digital signal processor, application specific integrated circuit, discrete logic, or any combination thereof. Analog or digital information representing audio, visual, or audiovisual breathing intervals may be stored in digital or analog form by storage media subsystem G and retrieved for purposes of generating audible, visual, or audiovisual signals for presentation to the user. Breathing signal information may also stored in memory as data and instruction sequences for purposes of synthesizing breathing signal by control subsystem C for purposes of presentation to the user.

An instructive method is also specified for use by respiratory care practitioners and care recipients.

Instructive Method for Reducing Sympathetic Predominance, and Consequent Positive Modifications to its Attendant Symptomology Hypertension:

1. A careful overview of care recipients health status and background are conducted.
2. A breath therapy strategy is developed and discussed between care recipient and practitioner.
3. The care recipient is instructed to assume a comfortable posture.
4. The care practitioner or care recipient attaches breathing cycle monitoring apparatus. This may be a discrete monitoring apparatus per the embodiment of FIG. 3 or an integrated apparatus per the embodiment of FIG. 6.
5. The care practitioner or care recipient assesses and records the present breathing cycle.
6. If appropriate, care practitioner or care recipient attaches blood pressure measurement apparatus and records present blood pressure readings.
7. Per terms of the breathing therapy developed in step 2, a training strategy is selected involving the selection of one or more breathing frequencies in descending order, for example, 18 breathing cycles per minute followed by 15 breathing cycles per minute. A decision is also made as to how long to train each breathing cycle.
8. The care practitioner or recipient turns on the breathing cycle timing generator and the recipient begins practice.
9. The care practitioner instructs the recipient to inhale on the first cue and exhale on the successive cue, inhaling and then exhaling on cue for the duration of the practice.
10. The care practitioner instructs the recipient to align the end of their exhalation and the beginning of their inhalation with the first signal and the end of their inhalation and the beginning of their exhalation with the second signal as closely as is comfortably possible.
11. The care recipient practices in this manner for the duration of the training period.
12. The care practitioner monitors the correctness and comfort of the recipient during the process.
13. At the end of the training session, the care practitioner instructs the recipient that they are to attempt to maintain this relatively slower rate of breathing throughout their daily activities.
14. As is appropriate, the care practitioner or care recipient once again assesses the blood pressure and records the results.
15. Over the course of time, with adequate adoption of the new breathing behavior, i.e. practice and incorporation in to daily life, the frequency of the breathing cycle is lowered with a corresponding decrease in blood pressure.
16. The objective is for the care recipient to reach the final objective of 1 breath in 11.76 seconds or 5 breaths in approximately 1 minute. This requires the recipient to inhale and exhale every 5.88 seconds. This also requires a certain “depth” in inhalation and exhalation.
17. Once the recipient is fully capable and comfortable with breathing at the target rate and depth, the formal modification phase is at an end and the maintenance phase begins.
18. The care practitioner instructs the care recipient that in order to maintain this breathing frequency continuous practice is required. This is necessary so that awareness of the breathing cycle remains and to prevent a gradual return to a higher breathing cycle frequency.
19. As is appropriate, the care practitioner instructs the care recipient to monitor and record their blood pressure on a regular basis.
20. In the acute case, where high blood pressure is of severe concern, the care practitioner fits the care recipient with the integrated training and monitoring apparatus of FIG. 5 and instructs the care recipient in the use thereof. This course of action may take place as early as step 3 if deemed appropriate.
21. In this case, breathing frequency is monitored on an ongoing basis during waking hours. If at any time the breathing frequency increases above a certain threshold or breathing depth decreases below a certain threshold, an alert is sounded. Depending on options selected, upon the alert an audible, visual, or audiovisual signal may begin automatically to which the care recipient is to synchronize their breathing. This signal continues until the breathing frequency and depth falls below specified thresholds. This has the effect of modifying the tendency toward sympathetic predominance in the moment. Relative to hypertension, the result being the maintenance of a relatively lower heartbeat rate and resultant blood pressure.

Those skilled in the art will recognize improvements and modifications to the preferred embodiments of the present invention. All such improvements and modifications are considered within the scope of the concepts disclosed herein and the claims that follow.

What is claimed:

1. The broad method of breathing therapy wherein the frequency of a care recipient's breathing cycle when in the state of rest or semi-activity is systematically reduced and the depth of the breathing cycle is systematically increased for the purpose of achieving balance of the autonomic nervous system, specifically reducing either acute or chronic sympathetic predominance, the ultimate goal being the realization and ongoing maintenance of 1 complete breathing cycle in 11.76 seconds or 5 complete breathing cycles in 58.8 seconds, the result being improved health, well being, and homeostasis.

2. The broad system facilitating the systematic reduction of breathing frequency and systematic increase in breathing depth for the purpose achieving balance of the autonomic nervous system, specifically reducing either acute or chronic sympathetic predominance, the result being improved health, well being, and homeostasis, the ultimate goal being the realization and ongoing maintenance of 1 complete breathing cycle in 11.76 seconds or 5 complete breathing cycle in 58.8 seconds, inclusive of breathing detection, breathing cycle timing generation, audio, visual, and audiovisual display, programmability, and control functions and aspects.

3. The method of claim 1, wherein 26 different breathing rhythms and associated intervals, specifically 30 breathing cycles per minute through 5 breathing cycles in 58.8 seconds, are employed in sequential fashion over a period of time to gradually bring a care recipient from a higher breathing frequency and related relatively shallow depth to a lower breathing frequency and relatively greater depth.

4. The system of claim 2, wherein 26 different breathing rhythms and associated intervals are provided in either audio, visual, or audiovisual format for purposes of synchronizing the breathing cycle for the purpose of gradually reducing the resting breathing frequency and increasing related breathing depth.

5. The system of claim 2 wherein, the 26 breathing rhythms and associated intervals are generated and stored on a various storage media, specifically compact disk (CD), digital video disk (DVD), digital tape, analog tape, flash memory, memory sticks, etc. for purposes of presentation to the care recipient.

6. The system of claim 2 wherein, the 26 breathing rhythms and associated intervals are generated via software or hardware processing techniques on any one of a microprocessor, digital signal processor, application specific integrated circuit, or discrete hardware, on any physical platform including personal computers, laptop computers, handheld computers, cell phones, or other devices.

7. The method of claim 1 wherein, the frequency of the breathing cycle is systematically reduced and the depth of the breathing cycle is systematically increased with the specific goal of reducing or eliminating the set of symptoms that are presently referred to as “hypertension”.

8. The system of claim 2, wherein the frequency of the breathing cycle is systematically reduced and the depth of the breathing cycle is systematically increased inclusive of breathing detection, breathing cycle timing generation, audio, visual, and audiovisual display, programmability, and control functions and aspects, with the specific goal of reducing or eliminating the set of symptoms that are presently referred to as “hypertension”,

9. The method of claim 1 wherein once realized, the care recipient continues to employ the present invention for the purpose of engaging in regular practice of breathing at the optimal interval of 11.76 seconds per breathing cycle for the purpose continuing to reinforce the optimal engram and to maintain fitness of the cardiopulmonary system and associated muscle groups.

10. The method of claim 1 wherein the broad method is applied for purposes of both prevention and cure of sympathetic predominance and the myriad of ailments and maladies to which it will ultimately be attributed.

11. The method of claim 1, wherein a care recipient's breathing frequency and depth are monitored on an ongoing basis, during normal walks of life, for conformance to target frequency and depth.

12. The system of claim 2, wherein an alert is provided when the care recipient's breathing frequency or breathing depth exceeds programmed limits.

13. The system of claim 2, wherein when an alert is initiated, a breathing rhythm is automatically generated and presented to the care recipient for purposes of synchronizing their breathing rhythm for the purpose of lower their breathing frequency and increasing their breathing depth so as to once again be within the specified target range as previously programmed.

14. The system of claim 2, wherein the generation and presentation of breathing cycle timing is provided on a continuously variable basis, starting at a specific point, for example 15 cycles per minute, and very gradually slowing to 5 breaths in 58.8 seconds with no pause between differing breathing cycle times.

15. The system of claim 2 wherein differing breathing cycle times are both sequentially and randomly accessible.

16. The system of claim 2, wherein breathing sensor, breathing cycle timing generator, and display elements may consist of discrete elements, for example the breathing cycle timing generator may be a CD player and the display device a set of headphones or alternatively a DVD player and a television set.

17. The system of claim 2, wherein the breathing sensor, the breathing cycle timing generator, display, and programmability interface are physically and functionally integrated.

18. The system of claim 2, wherein the breathing sensor is of a pulse detection or mechanical motion detection variety.

19. The system of claim 2, wherein functions of monitoring, breathing detection, breathing cycle timing generation, audio, visual, and audiovisual display, programmability, and control functions and aspects are assembled in a multiplicity of packaging variations.

20. The instructive method for care recipients and care practitioners for applying the preferred embodiments of the systems and methods of the present invention for purposes of reducing sympathetic predominance and optionally, its attendant symptomology “hypertension”.

21. The method of claim 1, wherein a care recipient's average heartbeat rate, specified in beats per minute, is assessed while the care recipient synchronizes their breathing cycle with:
a. An external reference signal of 11.76 seconds, thus yielding the optimal “target” average heartbeat rate, or alternatively,
b. Their heart rate variability rhythm, again yielding the optimal “target” average heartbeat rate, for purposes of monitoring and comparing actual average heartbeat rate in beats per minute with “target” average heartbeat rate in beats per minute.

22. The method of claim 21, wherein the care recipient's average heartbeat rate is monitored during normal daily activities and used as the basis of alerting the care recipient that their breathing frequency and resultant heartbeat rate is above target. The care recipient uses this information to consciously correct their breathing pattern, slowing down their rate of respiration and increasing their depth of respiration.

23. The system of claim 2, wherein an average heartbeat rate monitor is applied to the care recipient on an continual basis during normal walks of life, and more specifically, wherein programmability of the average heartbeat rate monitor is provided such that the “target” average heartbeat rate can be specified and continually compared against the actual average heartbeat rate.

24. The system of claim 2, wherein an alerting threshold is provided both in terms of:
a. heartbeats per minute over target average heartbeat rate, and
b. interval during which target average heartbeat rate is exceeded.

25. The system of claim 23, wherein an audible, visual, or sensory alert is generated when the actual average heartbeat rate exceeds programmed thresholds.
